(19) United States
(12) Patent Application Publication
(10) Pub. No.: US 2010/0173025 A1
(43) Pub. Date: Jul. 8, 2010

(54) FAT ABSORPTION INHIBITORY COMPOSITION

(75) Inventors: Yuki Katayanagi, Fujimino-shi (JP); Shigeru Hiramoto, Fujimino-shi (JP)

Correspondence Address: SUGHRUE MION, PLLC, 2100 PENNSYLVANIA AVENUE, N.W., SUITE 800, WASHINGTON, DC, 20037, US

(73) Assignee: NISSHIN PHARMA INC., Chiyoda-ku, Tokyo (JP)

(21) Appl. No.: 12/676,321

(22) PCT Filed: Sep. 4, 2008
(86) PCT No.: PCT/JP2008/065933

§ 371 Date: Mar. 3, 2010

(30) Foreign Application Priority Data
Sep. 4, 2007 (JP) 2007-229060

(51) Int. Cl. A61K 36/899; A61P 3/04; A61P 3/06; A61P 9/12; A61P 3/10

(52) U.S. Cl. 424/750

(57) ABSTRACT

The present invention provides a composition that exhibits favorable fat absorption inhibitory activity, is capable of being safely and easily taken continuously without side effects, and further is capable of preventing and/or improving conditions associated with excessive fat absorption such as obesity and hyperlipidemia. The present invention relates to a fat absorption inhibitory composition, which contains as an active ingredient a component that is collected as insoluble matter obtained by extracting wheat germ with water at 65° C. or lower and pH 5.0 or lower and treating the extract at 70° C. or higher and pH 6.0 or higher.

### TECHNICAL FIELD

The present invention relates to a fat absorption inhibitory composition having no or extremely low lipase inhibitory activity. More specifically, the present invention relates to a fat absorption inhibitory composition which contains, a wheat germ component as an active ingredient, does not cause side effects associated with lipase inhibitory activity since it has no lipase inhibitory activity and therefore can be safely and easily ingested.

### BACKGROUND ART

In recent years, along with lack of exercise and Westernized dietary habits, the percentage of those with obesity among the total population has been increasing. Obesity is a risk factor of lifestyle-related diseases in modern society. It has been pointed out that visceral fat accumulation would result in diabetes, hyperlipidemia, hypertension, insulin resistance, and the like. In addition to obesity, a combination of two or more diseases selected from among hyperlipidemia, hypertension and hyperglycemia is referred to as a metabolic syndrome. Individuals with metabolic syndromes are regarded as being classified into a high risk group with regard to cardiovascular diseases. The main factor causing obesity is excessive fat absorption exceeding fat consumption. However, this factor varies significantly among individuals. In addition, various factors such as genetic factors, social factors and home environment influence obesity. Thus, there are no effective methods for preventing and improving obesity. Mazindol, which is an appetite inhibitor, has been exclusively approved as a therapeutic agent against obesity in Japan. However, it can be used only for severe obesity. In addition, side effects such as tolerance, dependency, thirst, constipation, gastric distress and nausea/vomiting have been reported. In addition, orlistat, which has lipase inhibitory activity and acts to inhibit fat absorption via the intestinal tract, has been marketed as an obesity improving agent in foreign countries. However, it has been reported that the agent causes side effects such as passing of gas, steatorrhea, increased passing of stool, loose stool, diarrhea and abdominal pain. Those who use the agent cannot go out because they worry about having the sudden urge to have a bowel movement. This results in significant deterioration of quality of life (QOL), which is problematic.

For prevention/improvement of obesity, it is effective to continue adequate exercise and to reduce the intake calories through dietary restriction. However, excessive exercise and dietary restriction can be physically harmful. In addition, there is the concern of rebound reaction. Therefore, it is often difficult to carry out the above in daily living. Thus, it can be a very effective method for inhibiting in vivo absorption of diet-derived fat in a safe and healthy manner in order to prevent and treat obesity and related diseases.

Food products for specified health use capable of inhibiting fat absorption, which are non-pharmaceutical products, such as a globin protein degrading product capable of causing pancreatic lipase inhibition and thereby inhibiting fat absorption and diacylglycerol with poorly-digested/absorbed feature than fat have been commercially available. Further, novel materials with similar features have been actively screened. For instance, Patent Document 1 discloses a lipase inhibitor extracted from non-fat rice germ. In addition, Patent Document 2 discloses a lipase inhibitor containing, as an active ingredient, a wheat-germ-derived basic protein. Furthermore, Patent Document 3 discloses a lipase inhibitor containing, as an active ingredient, oolong theanine gallate obtained by polymerizing epigallocatechin gallate serving as a main catechin component of oolong tea with an enzyme.

However, the above lipase inhibitor does not have sufficient effects. In addition, it causes side effects associated with lipase inhibition activity such as passing of gas, steatorrhea, increased passing of stool, loose stool, diarrhea, and abdominal pain as in the case of orlistat. Consequently, it cannot be continuously digested.

### Patent Document 1: JP Patent Publication (Kokai) No. 7-25779 A (1995)

### Patent Document 2: JP Patent Publication (Kokai) No. 4-400839 A (1992)

### Patent Document 3: JP Patent Publication (Kokai) No. 2006-1909 A

### DISCLOSURE OF THE INVENTION

It is an object of the present invention to provide a composition which exhibits favorable fat absorption inhibitory activity, is capable of being safely and easily taken continuously without side effects, and is capable of preventing and/or improving conditions associated with excessive fat absorption such as obesity and hyperlipidemia.

In view of the above problems, the present inventors examined fat absorption inhibitory activity in plant extracts that are highly safe for edible use, and they found a material showing favorable fat absorption inhibitory activity in spite of having extremely low lipase inhibitory activity. This finding has led to the present invention.

Specifically, the present invention encompasses the following inventions.

(I) A fat absorption inhibitory composition, containing, as an active ingredient, an insoluble matter which is obtained by the following step, extracting wheat germ with water at 65° C. or lower and pH 5.0 or lower, and treating the extract under the following conditions (1) and/or (2): (1) pH 6.0 or higher; and (2) 70° C. or higher.
(II) The composition according to (I), which is used for preventing and/or improving conditions associated with excessive fat absorption.
(III) The composition according to (II), wherein the conditions associated with excessive fat absorption are obesity and/or hyperlipidemia.
(IV) The composition according to any one of (I) to (III), which has no or extremely low lipase inhibitory activity.

The present invention provides a safe fat absorption inhibitory composition that has favorable fat absorption inhibitory activity and has no side effects such as passing of gas, steatorrhea, increased passing of stool, loose stool, diarrhea or abdominal pain, which are observed in the case of a lipase inhibitor. The fat absorption inhibitory composition of the present invention comprises wheat germ for edible use as a raw material. Therefore, it has a good flavor and thus can be continuously taken with ease. Further, the fat absorption inhibitory composition of the present invention can be used for preventing and/or improving conditions associated with excessive fat absorption such as obesity and hyperlipidemia.

This description incorporates the contents of Japanese Patent Application No. 2007-229060, which is a priority document of the present application.

### BEST MODE FOR CARRYING OUT THE INVENTION

Preferred embodiments of the present invention are specifically described below.

In the present invention, the term “fat absorption inhibition” refers to inhibition or retardation of incorporation of ingested fat absorbed via the intestinal tract into the body. In addition, the term “fat absorption inhibition” also includes reduction of the fat or fatty acid level in blood and inhibition of an increase in fat in blood after fat ingestion. Examples of a method for evaluating fat absorption inhibition include a method for determining the level of fat in blood or stool after administering a quantified portion of fat into the intestinal tract by oral ingestion or the like, a method for determining the amount of body fat over time, and a method for determining the level of fat transferred from the mucosa side to the serosa side in an excised intestinal tract specimen.

In the present invention, the term “lipase inhibitory activity” refers to an effect of inhibiting the triglyceride degrading activity of pancreatic lipase mainly secreted in vivo from the pancreas. A fat component in food: i.e., triglyceride, is not absorbed in vivo as is, is absorbed as monoacylglycerol and fatty acid after hydrolyzation with lipase. Therefore, a component having lipase inhibitory activity can inhibit systemic absorption of fat; however, non-absorbed triglyceride remains in large amounts in the intestinal tract. This causes various side effects observed after administration of such component, leading to QOL deterioration. Examples of a method for evaluating the lipase inhibitory activity of a test substance include: a method comprising incubating lipase with triglyceride serving as a substrate in the presence of a test substance and determining the level of fatty acid released as a result of degradation; and a method comprising incubating lipase with triglyceride serving as a substrate and determining the level of remaining triglyceride.

In the present invention, the phrase “the composition having no or extremely low lipase inhibitory activity” refers to a lipase inhibitory rate of 20% or less and of preferably 15% or less, which is obtained by a method for determining the lipase inhibitory activity that is similar to the method used in Test Example 2 described below.

The fat absorption inhibitory composition of the present invention contains, as an active ingredient, a component obtained by purifying wheat germ serving as a raw material with a specific method (hereinafter, referred to as a wheat germ purified product). Herein, a preferred example of such a specific method is a method described in JP Patent Publication (Kokai) No. 10-218890 A (1998). Specifically, the method comprises: step A of extracting wheat germ with water at 65° C. or lower and pH 5.0 or lower; and step B of collecting insoluble matter by treating the obtained extract under the following conditions (1) and/or (2):(1) pH 6.0 or higher; and (2) 70° C. or higher.

Steps A and B are described in detail below.

### (Step A)

Water used in this extraction step may contain salts such as NaCl and/or KCl, ions, trace metals and the like as long as the pH and the temperature fall within the predetermined ranges without preventing this step. Preferred water includes ion-exchange water, distilled water, pure water, ultrapure water, tap water and well water. Examples of an acidic substance used for adjusting the pH to 5.0 or lower include: inorganic acids such as hydrochloric acid, sulfuric acid and phosphoric acid; organic acids such as acetic acid, propionic acid, butyric acid, oxalic acid, malonic acid, succinic acid, fumaric acid, maleic acid, malic acid, lactic acid, tartaric acid, citric acid and benzoic acid; and acid water. In view of safety, it is preferable to use edible acid such as citric acid or lactic acid. Herein the term “acid water” refers to water obtained on the anode side upon electrolysis of water in the presence of an electrolyte such as dilute sulfuric acid or a common salt, and it is also referred to as oxidized water or anode water.

The extraction temperature is 65° C. or lower, preferably 15° C. to 65° C., more preferably 30° C. to 60° C. and particularly preferably 45° C. to 55° C. Extraction at step A is carried out at 65° C. or lower and acidic conditions such as pH 5.0 or lower and preferably pH 3.0 to 5.0 for generally 5 to 20 hours and preferably 10 to 18 hours.

Wheat germ can be directly subjected to step A. The form of wheat germ is not particularly limited, and therefore wheat germ may be in the form of a powder product, a disrupted product, or a particle product. However, in view of ease of treatment, a powder product is preferable. A commercially available wheat germ product such as non-fat germ can be used.

In addition, it is possible to carry out, as pretreatment of step A, enzymatic degradation treatment of starch, protein, and hemicellulose. An example of enzymatic degradation treatment of starch is amylase treatment. An example of enzymatic degradation treatment of protein is protease treatment. An example of enzymatic degradation treatment of hemicellulose is cellulase treatment. However, pretreatment is not limited thereto. When amylase treatment, protease treatment, cellulase treatment, or a similar treatment is carried out, it is preferable to carry out treatment under conditions generally used for enzyme treatment; that is to say, conditions including the optimum temperature and pH for a relevant enzyme. However, the conditions are not limited to such examples.

In step A, if the temperature is 70° C. or higher and/or the pH is 6.0 or higher, a component of interest serving as an active ingredient in the present invention is formed into insoluble matter. Such insoluble matter precipitates, resulting in reduction of extraction efficiency in some cases. Therefore, it is necessary to precisely control the temperature and the pH in the extraction step. However, it is acceptable to adjust the temperature to 70° C. or higher or the pH to 6.0 or higher before or after step A for the purposes of removal of the aforementioned fat or oil component, enzyme treatment, sterilization, and the like. In addition, it is preferable to remove insoluble matter or impurities in the extracted solution before applying the extracted solution to step B.

### (Step B)

When the extracted solution obtained in step A is adjusted to: (1) a temperature of 70° C. or higher; and/or (2) a pH of 6.0 or higher, a component of interest serving as an active ingredient in the present invention is formed into insoluble matter. Therefore, it is possible to readily purify an active ingredient of the fat absorption inhibitory composition of the present invention by separating the insoluble matter from the liquid portion. In step B, both of or either one of the following conditions can be applied: (1) a temperature of 70° C. or higher; and (2) a pH of 6.0 or higher. In addition, if both conditions are applied, the order of the conditions is not particularly limited. Further, both conditions may be simultaneously satisfied. The temperature is generally 70° C. to 100° C. and preferably 80° C. to 95° C. The pH is generally 6.0 to 12.5, preferably 6.5 to 10.0, and more preferably 7.0 to 9.0. The pH can be adjusted with basic substances such as sodium hydroxide and potassium hydroxide.

As a method for separating and collecting the composition of the present invention in the form of insoluble matter, a known technique can be used. Examples thereof include filtration, centrifugation, and filter pressing. In addition, regarding the fat absorption inhibitory composition of the present invention, the above insoluble matter can be directly dried for use. However, according to need, the insoluble matter can be dissolved in an organic solvent or an acidic solution to result in a solution. Then, it can be dried with an excipient such as dextrin, lactose, or starch by a known method via lyophilization, spray drying, spray cooling, or drum drying.

The present inventors have found that the above wheat germ purified product has fat absorption inhibitory activity and therefore can prevent and/or improve conditions associated with excessive fat absorption. Specifically, the fat absorption inhibitory composition of the present invention containing, as an active ingredient, the above wheat germ purified product can be used as a composition capable of preventing and/or improving conditions associated with excessive fat absorption. Conditions associated with excessive fat absorption include obesity, hyperlipidemia, diabetes, hypertension, and metabolic syndromes. Particularly preferably, the fat absorption inhibitory composition of the present invention is used for preventing and/or improving obesity and/or hyperlipidemia.

The fat absorption inhibitory composition of the present invention is effective for preventing and/or improving the above conditions such as obesity, hyperlipidemia, diabetes, hypertension, and metabolic syndromes induced by fat absorption and subsequent fat accumulation, and particularly, conditions that are not associated with lipase activity, conditions that cannot be prevented and/or improved even by inhibiting lipase activity, and conditions for which it is not preferable to inhibit lipase activity. In addition, the fat absorption inhibitory composition of the present invention has no or extremely low lipase inhibitory activity. Therefore, it is advantageous in that side effects associated with lipase inhibitory activity such as passing of gas, steatorrhea, increase passing of stool, loose stool, diarrhea, and abdominal pain are not induced.

In the present invention, prevention of conditions includes inhibition or retardation of the onset of conditions and prevention of recurrence of conditions. In the present invention, improvement of conditions includes curing such conditions and preventing their progress.

Objects for administration of the composition of the present invention are preferably mammals. The term “mammals” used herein refers to warm-blooded vertebrates, for example, primates such as humans and monkeys; rodents such as mice, rats and rabbits; pet animals such as dogs and cats; and livestock such as bovines, horses and swines. The composition of the present invention is preferably administered to primates, and particularly preferably to humans.

The fat absorption inhibitory composition of the present invention which contains the above wheat germ purified product as an active ingredient is not particularly limited. However, it can be prepared as a pharmaceutical composition or a food composition.

When the fat absorption inhibitory composition of the present invention is administered to humans, it is administered at a dose of 0.001 g to 50 g based on the dry mass of the wheat germ purified product per day for an adult. The wheat germ purified product is highly safe because it is produced by using wheat germ as a raw material. Therefore, it is possible to increase the ingestion dose thereof. The daily ingestion dose can be used as a single dose or divided into several doses for ingestion.

When the fat absorption inhibitory composition of the present invention is prepared as a pharmaceutical composition, the above wheat germ purified product may be used alone. However, in general, it is prepared as a formulation containing the wheat germ purified product and a preferred pharmaceutically acceptable carrier. The pharmaceutical composition may be orally or parenterally administered. For example, it may be administered intradermally, subcutaneously, intravenously, intra-arterially, intramuscularly, intraperitoneally or intranasally. It may be mixed with an additive, other known fat absorption inhibitor and the like unless the effects of the present invention are impaired.

Examples of dosage forms of the pharmaceutical composition include: oral formulations such as tablets, capsules, granules, powders, syrups, dry syrups, liquids and suspensions; and enteral formulations such as inhalers and suppositories. Of these, oral formulations are preferable.

Pharmaceutical compositions having such dosage forms can be prepared by mixing the above wheat germ purified product with commonly used additives such as excipients, disintegrators, binders, lubricants, surfactants, alcohol, water, aqueous polymers, sweeteners, flavoring substances and acidulants according to dosage forms. In addition, liquid formulations such as liquids and suspensions may be dissolved or suspended in water or other appropriate solvent immediately before administration. In addition, the surface may be coated with formulations such as tablets and granules by a known method.

In the pharmaceutical composition of the present invention, the content of the wheat germ purified product, which is an active ingredient, varies according to dosage forms. However, generally the content based on dry mass is 0.001% to 99% by mass and preferably 0.01% to 80% by mass. It is preferable that the dosage form allows the daily dosage to be controlled such that the above ingestion dose per day for an adult can be achieved.

When the fat absorption inhibitory composition of the present invention is prepared as a food composition, the form thereof is not particularly limited. It may be any form of food that can contain the wheat germ purified product, including health food, functional food and food for specified health use. Specific examples of the form of food for specified health use various comprise various formulations, for example, liquid diet such as enteral feeding products, tablets, sugar-coated tablets, chewable tablets, powders, capsules and granules. These can be produced by a method similar to that used for the pharmaceutical composition. Food products of the present invention include beverages. Specific examples of beverages include nutritional supplements (e.g., drinks), tea beverages (e.g., green tea, oolong tea, and black tea), soft drinks, jelly drinks, sport drinks, milk beverages, carbonated drinks, fruit juice drinks, lactobacillus beverages, fermented milk beverages, powder-type drinks, cocoa drinks, coffee drinks and purified water. Further, food products of the present invention may be prepared in the form of spreads (e.g., butter, jam, furikake which is condiments for boiled rice, and margarine), mayonnaise, shortening, custard cream, dressings, breads, boiled rice, noodles, pasta, miso soup, tofu, milk, yogurt, soup, sauce and sweets (e.g., biscuits, cookies, chocolate, candies, cakes, ice cream, chewing gum, and tablets).

A food product of the present invention can be produced by a general method by mixing the wheat germ purified product which is an active ingredient with other food materials that can be used for food or feed production, various nutrients various vitamins, minerals, amino acids, various forms of fat and oil, dietary fibers and various additives (e.g., taste components, sweeteners, acidulants such as organic acids, surfactants, pH adjusters, stabilizers, antioxidants, dyes and flavors). In addition, it is also possible to produce a food composition as the fat absorption inhibitory composition of the present invention by mixing the wheat germ purified product with a generally available food product.

When the fat absorption inhibitory composition of the present invention is used as a food composition, the content of a wheat germ purified product, which is an active ingredient, is generally 0.001% to 80% by mass, preferably 0.01% to 50% by mass, and more preferably 20% to 50% by mass on basis of dry mass, although it may vary according to food formulation. The daily ingestion dose can be used as a single dose or divided into several doses for ingestion. It is preferable that the dosage form allow the daily dosage to be controlled such that the aforementioned ingestion dose per day for an adult can be achieved.

According to the present invention, the term “food” comprises not only food for humans but also feed for livestock or racehorses and pet food. Feed is substantially identical to food except that it is used for animals. Therefore, descriptions regarding food used herein can apply to feed in a similar manner.

In addition to the above, further, the fat absorption inhibitory composition of the present invention may be mixed with other functional component, for example, conjugated linoleic acid, taurine, glutathione, carnitine, creatine, coenzyme Q, glucuronic acid, glucuronolactone, capsicum extract, ginger extract, cacao extract, guarana extract, garcinia extract, theanine, γ-aminobutyric acid, capsaicin, capsiate, various organic acids, flavonoids, polyphenols, catechins, xanthine derivatives, non-digestible oligosaccharides such as fructooligosaccharide, and polyvinylpyrrolidone. The content of such a functional component to be mixed can be adequately determined depending on the desired ingestion dose and the type of component. However, the content in the fat absorption inhibitory composition of the present invention is generally 0.01% to 70% by mass and preferably 0.1% to 50% by mass.

The present invention is hereafter described in greater detail with reference to the following examples, although the technical scope of the present invention is not limited thereto.

### EXAMPLES

### Example 1

### Production of a fat Absorption Inhibitory Composition

2000 L of water and 4 kg of citric acid were added to 250 kg of non-fat wheat germ (produced by Nisshin Pharma Inc.) so as to adjust the pH to 5.0, followed by agitation for dispersion of non-fat wheat germ. Then, 1250 g of a starch degrading enzyme (liquid enzyme T; produced by HBI Enzymes Inc.) was added thereto, followed by heating. The resultant was maintained at 85° C. for 10 minutes and then cooled to 50° C. Thereafter, 1250 g of a starch degrading enzyme (liquid enzyme T) and 630 g of a protein degrading enzyme (protease M; produced by Amano Enzyme Inc.) were added thereto. The resulting mixture was subjected to a reaction at pH 5.0 and 50° C. for 14 hours (step A). The mixture was treated at 90° C. for 10 minutes for inactivating enzyme and cooled to 60° C. The composition of the present invention can be solubilized by such cooling step. Therefore, insoluble matter was removed with a centrifuge and activated charcoal was added to the supernatant, followed by agitation. Then, filtration was carried out by using diatomaceous earth as a filtration aid, followed by vacuum concentration of the filtrate. The concentrated solution obtained as an extract (Brix 35) was adjusted to 85° C. for formation of insoluble matter (step B). The obtained insoluble matter was collected by filtration while being maintained at 85° C., followed by drying under reduced pressure.

As described above, 3.85 kg of a wheat germ purified product in a dry solid form was obtained as the composition of the present invention. The color of the obtained composition was white and the product had substantially no taste and no smell.

### Example 2

### Production of a fat Absorption Inhibitory Composition

1500 kg of non-fat wheat germ (produced by Nisshin Pharma Inc.), 12000 L of ion-exchange water, 131 kg of anhydrous citric acid and 15 kg of a starch degrading enzyme (liquid enzyme T; produced by HBI Enzymes Inc.) were added to a large pot, followed by agitation. The mixture was adjusted to 85° C., followed by dispersion for 10 minutes. Next, the mixture was cooled to 50° C. 15 kg of a starch degrading enzyme (liquid enzyme T; produced by HBI Enzymes Inc.) and 3.6 kg of a protein degrading enzyme (protease M; produced by Amano Enzyme Inc.) were added thereto. The mixture was adjusted to pH 4.0 and subjected to a reaction at 50° C. for 14 hours (step A). After completion of the reaction, the mixture was treated at 90° C. for 10 minutes for inactivating enzyme, followed by cooling to 60° C. Activated charcoal was added thereto for adsorption of insoluble matter at 60° C. for 1 hour. The liquid mixture was filtrated via filter pressing and the filtrate was cooled to 35° C. NaOH was added to the obtained filtrate in an extracted solution form so as to result in pH 9.0 for formation of insoluble matter (step B). The obtained insoluble matter was collected with a centrifuge, followed by vacuum drying. Accordingly, a powder was obtained by vacuum drying. The powder was suspended in distilled water in a mass 5-fold greater than that of the powder. Hydrochloric acid was added thereto during agitation so as to result in pH 3.0 for dissolution of the powder. A slight amount of insoluble matter was removed by centrifugation, followed by lyophilization. As a result, 7.19 kg of a wheat germ purified product in a dry solid form was obtained as the composition of the present invention. The color of the composition was slightly yellow and the product had substantially no taste and no smell. The obtained composition was subjected to component analysis. The results are as follows: carbohydrate: 0.28% by mass; protein: 0.47% by mass; and lipid: 11.9% by mass.

### Example 3

### Production of a fat Absorption Inhibitory Composition

50 g of the composition obtained in Example 2 was added to a solution consisting of chloroform: methanol=5:1 (volume ratio), followed by agitation for 2 hours. Then, a filtrate was obtained using non-fat cotton. The filtrate was dried such that 6.0 g of a colorless oily matter was obtained as the composition of the present invention.

### Example 4

### Production of a fat Absorption Inhibitory Composition

1500 kg of non-fat wheat germ (produced by Nisshin Pharma Inc.), 12000 L of ion-exchange water, 131 kg of anhydrous citric acid and 15 kg of a starch degrading enzyme (liquid enzyme T; produced by HBI Enzymes Inc.) were added to a large pot, followed by agitation. The mixture was adjusted to 85° C., followed by dispersion for 10 minutes. Next, the mixture was cooled to 50° C. 15 kg of a starch degrading enzyme (liquid enzyme T; produced by HBI Enzymes Inc.) 3.6 kg of and a protein degrading enzyme (protease M; produced by Amano Enzyme Inc.) were added thereto. The mixture was adjusted to pH 4.0 and subjected to a reaction at 50° C. for 14 hours (step A). After completion of the reaction, the mixture was treated at 90° C. for 10 minutes for inactivating enzyme, followed by cooling to 60° C. Activated charcoal was added thereto for adsorption of insoluble matter at 60° C. for 1 hour. The liquid mixture was filtrated by filter pressing and the filtrate was cooled to 35° C. NaOH was added to the obtained filtrate as an extracted solution form so as to result in pH 8.0 for formation of insoluble matter (step B). The obtained insoluble matter was collected with a centrifuge and dispersed in a citric acid solution (pH 3.1). Filtration was carried out to remove a slight amount of insoluble matter. Dextrin was added to the resultant, followed by spray drying. Accordingly, 273 kg of a wheat germ purified product in a dry solid form was obtained as the composition of the present invention.

### Example 5

### Production of a fat Absorption Inhibitory Composition

100 L of water was added to 12.5 kg of non-fat wheat germ (produced by Nisshin Seifun). Lactic acid was added thereto so as to result in pH 4.0, followed by agitation at 30° C. for 4 hours for extraction (step A). Insoluble matter was removed by a centrifuge. The pH of the obtained aqueous solution was adjusted with NaOH to 7.5 for formation of insoluble matter (step B). The obtained insoluble matter was collected using a centrifuge. Thus, a wheat germ purified product in a paste form was obtained as the composition of the present invention.

### Test Example 1

### Determination of fat Absorption Inhibitory Activity

A test was conducted in a manner such that 10 mL/kg weight of Intralipid 20% (produced by TERUMO) was orally administered to rats (SD, male) followed by blood sampling over time. The test was performed to determine the amount of fat transferred to blood via absorption through the intestinal tract. The rats were forcibly subjected to oral administration (1 g/kg weight) of the compositions of the present invention obtained in Examples 2, 3 or 4 with Intralipid. Blood obtained from the caudal vein was conducted before and 2 hours after administration. The triglyceride level in blood was determined using a triglyceride determination kit (produced by Wako Pure Chemical Industries, Ltd.). Only Intralipid was administered to a control group without administration of the compositions of the present invention. Each group consisting of 10 mice was tested. Results were evaluated based on the triglyceride levels determined before administration of Intralipid, the triglyceride levels determined 2 hours after administration of Intralipid, and the AUC values determined up to 5 hours after administration of Intralipid. Table 1 lists the results.

TABLE 1

| | Triglyceride level in blood (mg/dL) | | | |
|---|---|---|---|---|
| Administration group | Example 2 | Example 3 | Example 4 | Control |
| Before administration | 66.2 | 71.0 | 57.2 | 75.8 |
| 2 hours after administration | 179.3 | 88.8 | 200.9 | 247.9 |
| AUC (5 h) | 398.1 | 111.9 | 389.1 | 499.7 |

Based on table 1, it is found that the compositions of the present invention can obviously inhibit absorption of triglyceride into blood.

### Test Example 2

### Determination of Lipase Inhibitory Activity

Ten micro litter of a solution containing the composition of the present invention obtained in Example 2 or 3 (diluted with a buffer to a final concentration of 0.01 or 0.1 mg/mL) or 10 μL of a solution containing the composition of the present invention obtained in Example 4 (at a final concentration of 1 mg/mL) was added to 80 μL, of a substrate emulsion (3.0 g of obtained by adding olive oil to 100 mL of a buffer described below, followed by sonication). The resultant was allowed to stand at 37° C. for 10 minutes. Ten micro litter of a lipase solution (diluted with a buffer to a concentration of lipase (produced by Sigma) of 1.0 mg/mL) was added thereto. The mixture was reacted with an enzyme reaction at 37° C. for 30 minutes. After completion of the reaction, 0.5 mL of a copper reagent (an aqueous solution containing 0.45 mol of triethanolamine, 0.05 mol of acetic acid, 3.8% by mass of copper sulfate 5-hydrate and 20% by mass of NaCl in 100 mL thereof) was added to the mixture. Further, 1.5 mL of an extraction reagent consisting of chloroform: n-heptane: methanol=49:49:1) was added thereto, followed by sufficient mixing for 30 seconds. The resultant was centrifuged at 3000 rpm for 10 minutes in order to collect 0.5 mL of the supernatant. A coloring solution was added to the supernatant for determination of an absorbance at 480 nm. As a positive control, 0.1 or 1 mg/mL of purothionin (produced by Sigma) at final concentration), which is a wheat-derived basic protein with lipase inhibitory activity (see, JP Patent Publication (Kokai) No. 3-284627 A (1991)), was used. Results were calculated by Equation 1 described below with absorbance of the following: the composition of the present invention; a blank replaced the lipase solution with a buffer; and a control replaced a solution containing the composition of the present invention with a buffer. Table 2 lists the results.

### [Buffer Composition]

Taurochenodeoxycholate: 1 mM

Sodium taurocholate: 9 mM

Cholesterol: 0.1 mM

L-α-phosphatidylcholine: 0.8 mg/mL

Calcium acetate: 1 mM

Tris (hydroxymethyl) aminomethane: 100 mM

[Equation 1]

Inhibition rate (%)=[1−[absorbance of the composition of the present invention−blank absorbance]/[control absorbance−blank absorbance]]

TABLE 2
Lipase inhibition rate (%)

| | Example 2 | Example 3 | Example 4 | Positive control |
|---|---|---|---|---|
| 0.01 mg/mL | −1 | −3 | — | — |
| 0.1 mg/mL | −3 | 11 | — | 76 |
| 1 mg/mL | — | — | −0.2 | 96 |

The results listed in table 2 indicate that the composition of the present invention exhibits no lipase inhibitory activity. Therefore, it is found that the wheat germ purified product of the present invention has components that completely differ from those of conventional lipase inhibitors.

### Example 6

### Production of Tablets

84 g of the fat absorption inhibitory composition obtained in Example 1, 10 g of crystalline cellulose (Asahi Kasei Corporation), and 5 g of polyvinylpyrrolidone (BASF) were mixed. 30 mL of Ethanol was added thereto for granule production by a general wet method. The obtained granules were dried. Then, 1.2 g of magnesium stearate was added thereto to obtain a granule powder for tableting. Tableting was carried out using a tableting machine such that 100 tablets (1 g per tablet) were produced.

### Example 7

### Production of Granules

100 g of the fat absorption inhibitory composition obtained in Example 2, 100 g of lactose (DMV) and 40 g of crystalline cellulose (Asahi Kasei Corporation) were mixed together. 130 mL of the mixture and ethanol were added to a kneader, followed by kneading for 5 minutes by a general method. After completion of kneading, the product was sieved (10 mesh) and dried in a drying apparatus at 50° C. After drying, 240 g of granulating was carried out to obtain granules.

### Example 8

### Production of Bread

Wheat flour (hard wheat flour) (166 g) and dry yeast (2 g) were mixed together. Independently, 2 g of the fat absorption inhibitory composition obtained in Example 1, 25 g of sugar, 3 g of common salt and 6 g of non-fat milk were dissolved in 70 g of warm water. A chicken egg was added thereto, followed by sufficient mixing. The resultant was added to the above mixture containing wheat flour and dry yeast. The resulting mixture was sufficiently kneaded by hand. Then, approximately 40 g of butter was added thereto, followed by sufficient kneading. Accordingly, 20 bread dough pieces in the form of rolls were prepared. Next, these bread dough pieces were subjected to fermentation. Thereafter, beaten egg was applied to the surface of each piece. The pieces were baked in an oven at 180° C. for approximately 15 minutes. Thus, rolls were prepared. The rolls were excellent in terms of appearance, taste and texture.

### Example 9

### Production of a Pasta Sauce

150 g of a meat sauce for pasta for a single person was poured into a pot. One gram of the fat absorption inhibitory composition obtained in Example 2 was added thereto, followed by heating. A pouch was filled with the sauce. The pouch was hermetically sealed with nitrogen substitution, followed by sterilization at 121° C. for 15 minutes. Thus, a pouch of pasta meat sauce was obtained.

All publications, patents, and patent applications cited herein are incorporated herein by reference in their entirety.

1. A fat absorption inhibitory composition containing, as an active ingredient, an insoluble matter which is obtained by the following step,
extracting wheat germ with water at 65° C. or lower and pH 5.0 or lower, and
treating the extract under the following (1) and/or (2) conditions:
(1) pH 6.0 or higher; and
(2) 70° C. or higher.

2. The composition according to claim 1, which is used for preventing and/or improving conditions associated with excessive fat absorption.

3. The composition according to claim 2, wherein the conditions associated with excessive fat absorption are obesity and/or hyperlipidemia.

4. The composition according to any one of claims 1 to 3, which has no or extremely low lipase inhibitory activity.
